(19) United States
(12) Patent Application Publication
(10) Pub. No.: US 2023/0063803 A1
(43) Pub. Date: Mar. 2, 2023

(54) SYSTEMS AND METHODS FOR MONITORING MEDICATION USAGE

(71) Applicant: LUMIO HEALTH INC., San Jose, CA (US)

(72) Inventors: Aakash Kumar AGARWAL, San Jose, CA (US); Mark YEGHIAZARIAN, El Cajon, CA (US); Kumar NADHAN, Foster City, CA (US)

(21) Appl. No.: 17/459,386

(22) Filed: Aug. 27, 2021

(51) Int. Cl. G16H 20/13; G16H 40/67; G16H 40/20; H04W 12/06; H04W 4/12; A61F 9/00

(52) U.S. Cl. CPC G16H 20/13; G16H 40/67; G16H 40/20; H04W 12/06; H04W 4/12; A61F 9/0008; G06Q 30/0633

(57) ABSTRACT

Various embodiments of the present disclosure provide systems and methods for managing medication usage. The method performed by a server system includes receiving a plurality of user inputs from a medication monitoring application available in a user device associated with a user. The plurality of user inputs includes an initial medication volume present in a medication dispenser associated with the user, a medication regimen, and dosage schedules of the medication. The method includes tracking a medication usage of the user through the medication dispenser based at least on the plurality of user inputs. The method further includes transmitting an alert message to the user and at least one authorized user of the medication monitoring application to whom the user authorized to share information related to the medication usage based at least on detection of a plurality of medication dispensing events.

### TECHNICAL FIELD

The present disclosure relates to managing medication usage of patients and, more particularly relates, to systems and methods for managing the medication usage of the patients and providing alerts and/or reminders to at least the patients, and caregiver(s) and health care professional(s) of the patients based on detecting non-compliance events.

### BACKGROUND

Pharmaceutical products are prescribed by physicians for treating patients suffering from an infection or other medical conditions. The pharmaceutical products may be in form of a topical spray, topical gel or ointment (for absorption through the skin), pills or tablets, dissolvable strips, and the like. Some of these pharmaceutical products are applicable for treating the patient suffering from an infection or other medical conditions involving an eye. For instance, a physician prescribes a medicated solution (or eye drops) for administering to the eyes of the patient. These medications are effective at treating the infection or condition of the patient when administered as prescribed.

Patients need to continuously monitor and track the regimen for taking appropriate medication on schedule. However, the patients may find it difficult to maintain a course of treatment by taking medication on the schedule. Additionally, complying with prescribed medication dosage requirements may be complicated in some scenarios, for example, a single drug may require a different amount of dosage guidelines at different times. Due to non-compliance with their regimens, the patients require additional and often more expensive care, for example, dedicated caregivers, nursing homes, living facilities, advanced medical procedures and therapies, and the like to help the patients follow their regimens to improve health.

Therefore, there is a need for techniques to manage the medication regimens of the patients and reduce the non-compliance in following their regimens to improve health care outcomes, in addition to providing other technical advantages.

### SUMMARY

Various embodiments of the present disclosure provide systems and methods for managing medication usage.

In an embodiment, a computer-implemented method is disclosed. The computer-implemented method performed by a server system includes receiving a plurality of user inputs from a medication monitoring application available in a user device associated with a user. The plurality of user inputs includes an initial medication volume present in a medication dispenser associated with the user, a medication regimen, and dosage schedules of the medication. The method includes tracking a medication usage of the user through the medication dispenser based at least on the plurality of user inputs. The method further includes transmitting an alert message to the user and at least one authorized user of the medication monitoring application to whom the user authorized to share information related to the medication usage based at least on detection of a plurality of medication dispensing events.

In another embodiment, a server system is disclosed. The server system includes a communication interface, memory storing executable instructions, and a processor operatively coupled with the communication interface and the memory. The processor is configured to execute the executable instructions to cause the server system to at least receive a plurality of user inputs from a medication monitoring application available in a user device associated with a user. The plurality of user inputs includes an initial medication volume present in a medication dispenser associated with the user, a medication regimen, and dosage schedules of the medication. The server system is caused to track a medication usage of the user through the medication dispenser based at least on the plurality of user inputs. Further, the server system is caused to transmit an alert message to the user and at least one authorized user of the medication monitoring application to whom the user authorized to share information related to the medication usage based at least on detection of a plurality of medication dispensing events.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1 illustrates an example representation of an environment related to at least some example embodiments of the present disclosure;

FIG. 2 illustrates a simplified block diagram of a server system used for monitoring medication usage associated with a user, in accordance with one embodiment of the present disclosure;

FIGS. 3A-3F collectively, represent example representations of user interfaces (UIs) displayed to the user for managing the medication usage, in accordance with an embodiment of the present disclosure;

FIGS. 4A-4B collectively, represent example representations of user interfaces (UIs) displayed to a caregiver of the user for managing the medication usage, in accordance with an embodiment of the present disclosure;

FIGS. 5A-5C collectively, represent example representations of user interfaces (UIs) displayed to a health-care professional of the user for managing the medication usage, in accordance with an embodiment of the present disclosure;

FIG. 6 illustrates a flow diagram of a computer-implemented method for managing the medication usage of the user and providing alerts upon occurrence of a plurality of medication dispensing events, in accordance with an embodiment of the present disclosure;

FIG. 7 is a simplified block diagram of a control unit of a medication dispenser, in accordance with one embodiment of the present disclosure; and

FIG. 8 is a simplified block diagram of an electronic device capable of implementing various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted to not obscure the embodiments herein unnecessarily. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

Various example embodiments of the present disclosure provide systems and methods for managing medication usage of the patients. At least one embodiment of the present disclosure provides a server system that provides alerts to the patients, and caregiver(s) and health care professional(s) of the patients based on detecting non-compliance in taking the medication at the prescribed schedule.

In an embodiment, the server system is configured to receive a plurality of user inputs from a medication monitoring application available in a user device associated with a user (i.e. patient). The plurality of user inputs includes an initial medication volume present in a medication dispenser associated with the user, a medication regimen, and dosage schedules of a medication. It should be noted that the medication dispenser is communicably coupled with the user device. The server system is configured to communicate with the application through a control unit associated with the medication dispenser. Specifically, the server system tracks a medication usage of the user through the medication dispenser based at least on the plurality of user inputs. Based on detecting a plurality of medication dispensing events, the server system transmits an alert message to the user and at least one authorized user of the medication monitoring application to whom the user authorized to share information related to the medication usage. In one scenario, the server system may transmit the alert message to the user at a threshold time, prior to a dosage schedule of the medication for reminding the user to take the medication. In another scenario, the server system may transmit the alert message to at least the user and the at least one authorized user post the dosage schedule of the medication, if the dosage schedule has passed and upon detection of non-compliance with the dosage schedules of the medication associated with the user. For instance, the at least one authorized user of the application to whom the user authorized to share the information related to the medication usage is one of a health care professional and caregiver(s) of the user.

Further, the server system is configured to receive a quantifiable information related to a time stamp of the medication intake, and a volume of the medication dispensed from the medication dispenser from the control unit, upon intake of the medication at each dosage schedule. In this scenario, the server system computes parameters related to a medication-instilling efficiency, a medication volume in the medication dispenser after each dispensing, and number of days remaining of the medication. Thereafter, the server system updates the parameters in the application for allowing the user and the authorized users to monitor the medication usage of the user. In some embodiments, the server system is configured to send a notification to the user and the authorized users based at least on determining the number of days remaining of the medication is less than a predefined threshold value, for indicating the user and the authorized users about a refill status of the medication.

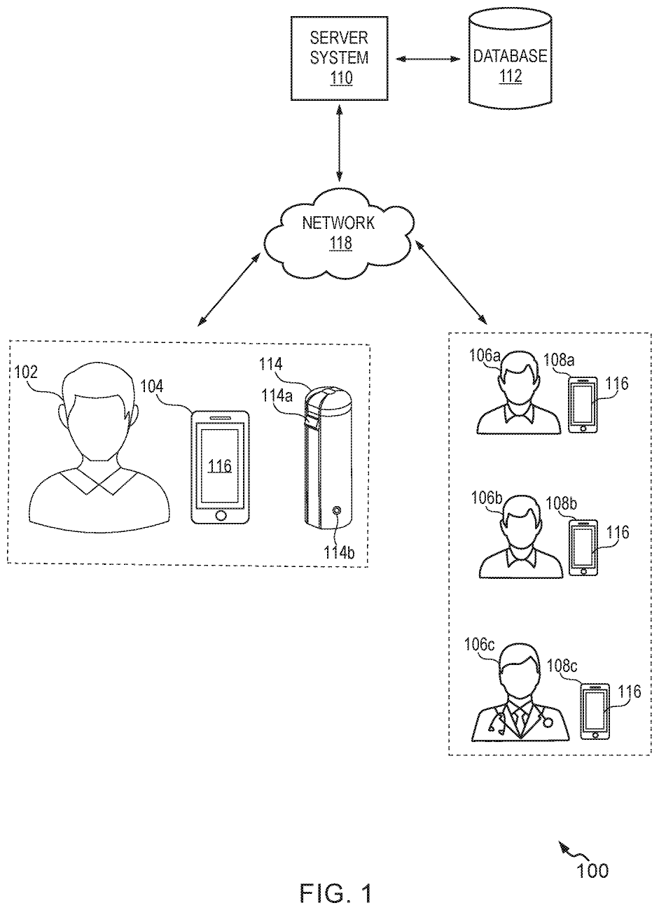

Various embodiments of the present invention are described hereinafter with reference to FIG. 1 to FIG. 8.

FIG. 1 illustrates an example representation of an environment 100 related to at least some example embodiments of the present disclosure. Although the environment 100 is presented in one arrangement, other arrangements are also possible where the parts of the environment 100 (or other parts) are arranged or interconnected differently. The environment 100 generally includes a user device 104 associated with a user 102, at least one authorized user 106a, 106b, and 106c associated with user devices 108a, 108b and 108c, respectively, a server system 110, a database 112, each coupled to, and in communication with (and/or with access to) a network 118. Examples of the user device 104 and the user devices 108a-108c may include, without limitation, smart phones, tablet computers, other handheld computers, wearable devices, laptop computers, desktop computers, servers, portable media players, gaming devices, and so forth. Further, the user 102 is associated with a medication dispenser 114 containing a medication. In an example embodiment, the user 102 may be a patient who is diagnosed for infection in eyes of the user 102. The user 102 may be required to take appropriate dosages of medication (e.g., ophthalmic liquid medication) from the medication dispenser 114 at scheduled time. Furthermore, the at least one authorized user 106a-106c may be one of caregivers and health-care professionals of the user 102. For instance, the users 106a, and 106b may be caregivers of the patient and the user 106c may be the health-care professional of the patient. The authorized users 106a-106c monitor medication usage of the patient (i.e. the user 102) which is herein explained further in detail.

Various entities in the environment 100 may connect to the network 118 in accordance with various wired and wireless communication protocols, such as, Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, or any combination thereof. In some instances, the network 118 may include a secure protocol (e.g., Hypertext Transfer Protocol (HTTP)), and/or any other protocol, or set of protocols. In an example embodiment, the network 118 may include, without limitation, a light fidelity (Li-Fi) network, a local area network (LAN), a wide area network (WAN) (e.g., the Internet), a mobile network, a virtual network, and/or another suitable public and/or private network capable of supporting communication among two or more of the entities illustrated in FIG. 1, or any combination thereof.

In one embodiment, the user devices 104 and 108a-108c are equipped with an instance of a medication monitoring application 116 (hereinafter interchangeably referred to as ‘the application 116’). The application 116 is a set of computer executable codes configured to provide user interfaces (UIs) to the user 102 and the users 106a-106c for monitoring medication usage of the user 102 and providing alerts and/or reminders based on occurrence of a plurality of medication dispensing events which will be explained further in detail. The medication monitoring application 116 may be hosted and managed by the server system 110. In an embodiment, the server system 110 may provide the application 116, in response to a request received from each of the user devices 104 and 108a-108c via the network 118. In another embodiment, the application 116 may be factory-installed on the user devices 104 and 108a-108c. In another embodiment, each of the user devices 104 and 108a-108c may access an instance of the application 116 from the server system 110 for installing on the user devices 104 and 108a-108c using application stores associated with various operating systems.

In addition, the server system 110 should be understood to be embodied in at least one computing device in communication with the network 118, which may be specifically configured, via executable instructions, to perform as described herein, and/or embodied in at least one non-transitory computer-readable media.

In the illustrative embodiment, the server system 110 is configured to track the medication usage from the medication dispenser 114 through the application 116 installed in the user device 104 and provide alerts, reminders based on occurrence of the plurality of medication dispensing events. As such, the user 102 and the users 106a-106c (i.e. the caregivers and the health-care professionals of the user 102) are required to undergo a one-time registration process in the application 116 by providing inputs in their respective user devices 104 and 108a-108c. In an example scenario, each of the users 102 and 106a-106c may be provided with a user interface (UI) with an option to select a category (such as, a patient, a caregiver and a health-care professional), while registering for the application 116. Upon registration, the server system 110 authenticates each of the users 102 and 106a-106c using login credentials associated with the users 102 and 106a-106c for enabling the users 102 and 106a-106c to access the application 116.

The server system 110 is configured to receive a confirmation message from the user device 104 based on enabling a wireless communication (or pairing) between the application 116 installed in the user device 104 and the medication dispenser 114. More specifically, the medication dispenser 114 may be configured with a wireless transceiver circuitry (not shown in Figures) for enabling the wireless communication between the user device 104 and the medication dispenser 114.

Further, the server system 110 is configured to create a user profile for each of the users 102 and 106a-106c based upon successful registration. The user profile of each of the users 102 and 106a-106c may be stored in the database 112. It should be noted that creating the user profile is herein explained with reference to the user 102 (i.e. the patient), therefore it should not be taken to limit the scope of the invention. Upon registration, the user 102 may provide a plurality of user inputs in the application 116. The user inputs may include, but are not limited to, an initial medication volume present in the medication dispenser 114, medication's inherent physical properties, a medication regimen, and dosage schedules associated the medication, and the like. As such, the server system 110 created the user profile for the user 102 in the application 116 based at least on receipt of the user inputs. In a similar manner, the server system 110 creates the user profile for the users 106a-106c (i.e. the caregivers and the health-care professionals) based at least on receipt of the information from each of the users 106. It should be understood that the users 106a-106c are the authorized users of the application 116, upon successful registration.

The server system 110 is configured to track the medication usage of the user 102 from the medication dispenser 114 based at least on the user inputs (such as, the medication regimen and the dosage schedules of the medication). Further, the server system 110 creates a log of the medication usage associated with the user 102 in the application 116 periodically based at least on the medication dispensing events. In one scenario, the server system 110 is configured to transmit information related to the medication usage of the user 102 to the users 106a-106c (i.e. the caregivers and the health-care professionals). More specifically, the user 102 may provide inputs related to details of the users 106a-106c with whom the user 102 wants to share the information related to the medication usage. This enables the server system 110 to facilitate reminders, provide educational content, and enable communication with patients' caregivers and health-care professionals (i.e. the users 106) through the application 116.

As explained above, the server system 110 is configured to provide an alert message to the user 102 about the medication based at least on the user inputs. More specifically, the server system 110 may transmit the alert message to the application 116 via messaging protocols, for reminding the user 102 to intake the medication at a prescribed dosage schedule. It should be understood that the alert message is transmitted to the user 102, prior to the medication dispensing event or the prescribed dosage schedule for providing reminder to the user 102 to take the medication from the medication dispenser 114. Further, the server system 110 transmits the alert message post the dosage schedule, if the user 102 has missed taking the medication at the prescribed dosage schedule.

In an example scenario, the server system 110 may also transmit the alert message to the users 106a-106c using the messaging protocols, prior to the dosage schedule and post the dosage schedule, for providing reminder to the users 106a-106c about the medication intake of the user 102 as explained above.

The server system 110 is configured to receive a quantifiable information being transmitted from the medication dispenser 114, upon intake of the medication at the dosage schedules (or at the medication dispensing events). Based upon receipt of the quantifiable information, the server system 110 computes one or more parameters (or output) and updates in the application 116 which is explained further in detail. In other words, the server system 110 transmits the parameters or the output to the user devices 104 and 108a-108c for allowing the users 102 and 106a-106c to monitor the medication usage.

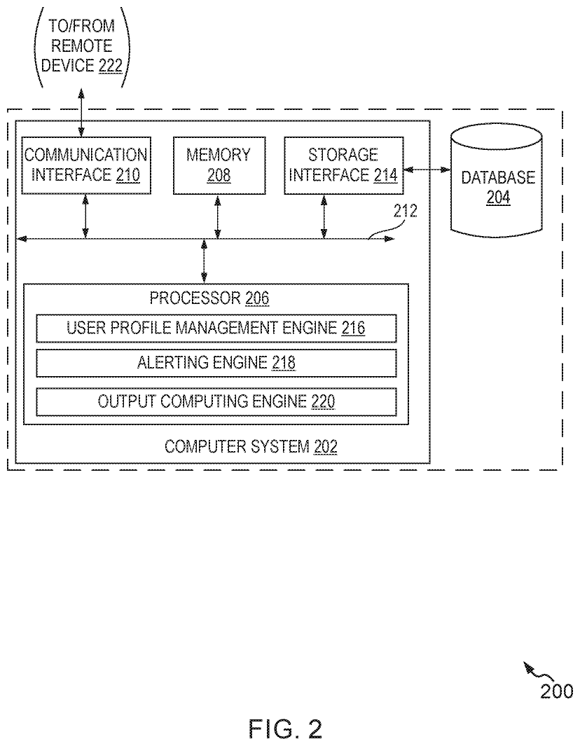

Additionally, the server system 110 is configured to detect non-compliance with the dosage schedules associated with the medication and transmit the alert message to at least the user 102 and the users 106a-106c. Detecting non-compliance with the dosage schedules and transmitting the alert message are further explained with reference to FIG. 2. The server system 110 is configured to detect remaining number of days of the medication and provides a notification to the user 102 and the users 106a-106c, if the remaining number of days of the medication is less than a predefined threshold value.

The number and arrangement of systems, devices, and/or networks shown in FIG. 1 are provided as an example. There may be additional systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of the environment 100 may perform one or more functions described as being performed by another set of systems or another set of devices of the environment 100.

FIG. 2 illustrates a simplified block diagram of a server system 200 used for monitoring medication usage associated with a user, such as the user 102, in accordance with one embodiment of the present disclosure. Examples of the server system 200 include, but are not limited to, the server system 110 as shown in FIG. 1.

The server system 200 includes a computer system 202 and a database 204. The computer system 202 includes at least one processor 206 for executing instructions, a memory 208, a communication interface 210 and a storage interface 214. The one or more components of the computer system 202 communicate with each other via a bus 212. The components of the server system 200 provided herein may not be exhaustive and the server system 200 may include more or fewer components than those depicted in FIG. 2. Further, two or more components may be embodied in one single component, and/or one component may be configured using multiple sub-components to achieve the desired functionalities.

In one embodiment, the database 204 is integrated within the computer system 202 and configured to store an instance of the application 116 and one or more components of the application 116. The one or more components of the medication monitoring application 116 may be, but not limited to, information related to user inputs, user profiles associated with the users 102 and 106a-106c, log of each action in the application 116, the alert message associated based on occurrence of the medication dispensing events, notifications, and the like. The computer system 202 may include one or more hard disk drives as the database 204. The storage interface 214 is any component capable of providing an access to the database 204, to the processor 206. The storage interface 214 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 206 with access to the database 204.

The processor 206 includes suitable logic, circuitry, and/or interfaces to execute computer readable instructions for performing security investigation and response analysis. Examples of the processor 206 include, but are not limited to, an application-specific integrated circuit (ASIC) processor, a reduced instruction set computing (RISC) processor, a complex instruction set computing (CISC) processor, a field-programmable gate array (FPGA), and the like. The memory 208 includes suitable logic, circuitry, and/or interfaces to store a set of computer readable instructions for performing operations. Examples of the memory 208 include a random-access memory (RAM), a read-only memory (ROM), a removable storage drive, a hard disk drive (HDD), and the like. It will be apparent to a person skilled in the art that the scope of the disclosure is not limited to realizing the memory 208 in the server system 200, as described herein. In some embodiments, the memory 208 may be realized in the form of a database server or a cloud storage working in conjunction with the server system 200, without deviating from the scope of the present disclosure. In some embodiments, the memory 208 may be realized in the form of a database server or a cloud storage working in conjunction with the server system 200, without deviating from the scope of the present disclosure.

The processor 206 is operatively coupled to the communication interface 210 such that the processor 206 is capable of communicating with a remote device 222 such as, the user devices 104 and 108a-108c, the medication dispenser 114, or with any entity connected to the network 118 as shown in FIG. 1.

It is noted that the server system 200 as illustrated and hereinafter described is merely illustrative of an apparatus that could benefit from embodiments of the present disclosure and, therefore, should not be taken to limit the scope of the present disclosure. It is noted that the server system 200 may include fewer or more components than those depicted in FIG. 2.

In one embodiment, the processor 206 includes a user profile management engine 216, an alerting engine 218 and an output computing engine 220. As such, the one or more components of the processor 206 as described above are communicably coupled with the application 116 and configured to monitor the medication usage, transmit the alert message, provide notifications related to refill status, share insights with the patients, caregiver(s), and health-care professional(s), and the like.

The user profile management engine 216 includes a suitable logic and/or interface for creating and managing user profiles of the users 102 and 106. Prior to creating the user profile, the user 102 and the users 106a-106c are required to undergo one-time registration process in the application 116. For instance, the user 102 may register for the application 116 by using an email ID, mobile phone number, and the like. As such, the user 102 is required to provide details used (e.g., email ID or mobile phone number) for registering to the application 116 to access one or more functionalities of the application 116 for monitoring the medication usage. The details used for registering to the application 116 correspond to login credentials.

In one scenario, the user 102 may be provided with the option for selecting the category of the user, while registering for the application 116. It should be understood that the server system 200 is configured to provide corresponding user interfaces (Uls) based on user selection of the category (such as the patient, the caregiver or the health-care professional). In an embodiment, the user 102 may be provided with a UI (not shown in Figures) depicting the option for selecting the category in the application 116, upon successful registration. Thus, a user (e.g., the user 102) may opt the category, while using the application 116. For instance, a user A may select the category as patient, while registering for the application 116. Also, the user A may provide details related to authorized users B and C of the application 116 who may be referred to as the caregiver and the health-care professional for the user A. Further, an authorized user D of the application 116 may opt the category as patient and provide details of the user A as the caregiver for the user D. In this scenario, the user A may switch between the categories (either to patient or caregiver) in the application 116 for monitoring the medication usage of the user A along with the user D.

Upon registration, the user 102 may provide the user inputs in the application 116. The user inputs may be, but not limited to, the initial medication volume present in the medication dispenser 114, medication's physical properties, the medication regimen, and the dosage schedules associated the medication, the details related to the caregivers and the health-care professional(s) (e.g., the users 106a, 106b and 106c) with whom the user 102 is authorized to share data related to the medication usage, the category opted by the user 102, and the like. Upon receipt of the user inputs, the user profile management engine 216 is configured to the user profile for the user 102 by populating the user inputs of the user 102. Further, the user profile of the user 102 is displayed to the authorized users (e.g., the users 106a-106c) of the application 116 based on users' selection (i.e. the user 102).

In a similar manner, the user profile management engine 216 is configured to create the user profiles for the users 106. In one example scenario, a caregiver (such as the user 106a) may provide user inputs in the application 116, upon successful registration. The user inputs associated with the caregiver (e.g., the user 106a) may be, but not limited to, name, age, contact details, and the like. In another example scenario, a health-care professional (such as the user 106c) may provide user inputs such as, but not limited to, name, profession details, contact details, and the like. As such, the user profile management engine 216 creates the user profile for the caregiver and the health-care professional in the application 116 based on the user inputs. Further, the user profile management engine 216 is configured to populate the information received from the patient (e.g., the user 102) such as the medication(s) of the patient, dosage schedules of the medication, and the like in the user profile of the caregiver and the health-care professional. For instance, the medication usage data is transmitted to the caregiver and the health-care professional (e.g., the users 106) based on receipt of the contact details (such as a username) of the users 106a-106c from the user 102. This enables the caregiver and the health-care professional (i.e. the users 106) to monitor the medication usage of the patient according to the dosage schedules.

In an embodiment, the caregiver or the health-care professional (e.g., the user 106b or the user 106c, respectively) of the user 102 may register the minimum period of dosing times (or the dosage schedules) through the application 116. Thus, the user 102 is intended to take the medication as updated by the user 106b or the user 106c.

Further, the server system 200 may receive information related to the medication dispenser 114, upon pairing the medication dispenser 114 with the user device 104. In one example embodiment, the medication dispenser 114 may include a unique code (e.g., a QR code), for allowing the user 102 to scan using the application 116. Upon scanning, the information appended to the unique code may be transmitted to the server system 200 through the application 116. The server system 200 is configured to authenticate the medication dispenser 114 as a genuine product based on the unique code and facilitates the wireless communication between the user device 104 and the medication dispenser 114. It should be understood that the wireless communication is enabled between the user device 104 and the medication dispenser 114 using short range communication protocols. Some non-limiting examples of the short range communication protocols are Bluetooth, Wireless-Fidelity (Wi-Fi), infrared, near field communication, ultraband, Zigbee, and the like. Additionally, the medication dispenser 114 is configured to communicate directly with the server system 200 and the user device 104 through cellular network.

In another example embodiment, the medication dispenser 114 may include a unique identification number printed on a housing (not shown in Figures) of the medication dispenser 114. In this scenario, the user 102 is required to enter the unique identification number of the medication dispenser 114 for pairing. The unique identification number may be generated and stored in the database 204 of the server system 200.

Additionally, the user 102 may check if the medication dispenser 114 is linked (i.e. paired) to the user device 104. Specifically, the user 102 may provide predefined number of press input (e.g., twice) on a button 114a of the medication dispenser 114. The processor 206 is configured to detect the press input (e.g., twice) in the medication dispenser 114 and displays a UI at the user device 104 for receiving confirmation from the user 102, if the button 114a was pressed twice. Based upon confirmation from the user 102, the data related to the medication usage will be automatically logged by the user profile management engine 216 in the user profile of the user 102. Further, in case of long-range network (i.e. the cellular network), the server system 200 may receive a signal from the user device 104 based on detecting the press input (e.g., twice) on the button 114a. In this scenario, the server system 200 would confirm the press input in the medication dispenser 114 and may display an acknowledgement on the user device 104.

The alerting engine 218 includes a suitable logic and/or interface for transmitting the alert message to at least the users 102 and 106a-106c based on detection of the medication dispensing events. The medication dispensing events include events such as the medication intake at the prescribed dosage schedules, missed dosage schedules, non-compliance with the dosage schedules, and the like, which are herein explained further in detail.

In one scenario, the server system 200 is configured to track the medication usage of the user 102 through the medication dispenser 114 based at least on the user inputs (i.e. the dosage schedules associated with the medication). In one scenario, the server system 200 is configured to receive user inputs related to a threshold time provided by the user 102 in the application 116. The threshold time may be a time, prior to a dosage schedule (or before a scheduled time) of the medication. As such, the alerting engine 218 is configured to transmit the alert message to the user device 104 at the predefined threshold time, prior to the dosage schedule of the medication, for reminding the user 102 to take the medication from the medication dispenser 114. For instance, the threshold time may be set for 10 minutes by the user 102 by providing inputs in the application 116 and the dosage schedule (or the scheduled time) of the medication may be at 3.00 PM every day. In this scenario, the alerting engine 218 transmits the alert message 10 minutes prior to 3.00 PM (or at 2.50 PM) on each day, for reminding the user 102 to take the medication. Additionally, the alerting engine 218 may transmit the alert message to the caregivers and the health-care professionals (e.g., the users 106b and 106c) of the user 102, to remind about medication of the user 102. In an embodiment, the threshold time may be predefined by the server system 200 and communicated to the user 102.

In another scenario, the alerting engine 218 is configured to transmit the alert message to at least the user 102 and the caregiver(s) (e.g., the user 106a) and health-care professional(s) (e.g., the user 106c) of the user 102, if the user 102 has missed to take the medication at the prescribed dosage schedule. In other words, the alerting engine 218 transmits the alert message to the user 102 and the caregiver(s) (e.g., the user 106a) and health-care professional(s) (e.g., the user 106c) of the user 102 post the dosage schedule, if the dosage schedule has passed. For instance, the user 102 may miss to take the medication at the dosage schedule and/or the prescribed scheduled time (e.g., 6.00 PM in the evening). In this scenario, the alerting engine 218 transmits the alert message to the user 102 and the users 106a-106c after the time (i.e. 6.00 PM) to take the medication, if the user 102 has missed to take the medication at 6.00 PM. In this case, the alerting engine 218 may send the alert message to the users 102 and 106a-106c after a time interval (e.g., 6 hours). As such, the server system 200 continuously monitors the medication intake by the user 102 from onset of the prescribed dosage schedule and until expiry of the time interval (e.g., 6 hours).

In yet another scenario, the alerting engine 218 is configured to send the alert message to the user 102 and the caregiver(s) and the health-care professional(s) (e.g., the user 106b and the user 106c, respectively) of the user 102 based on detecting the non-compliance with the dosage schedules. Some example scenarios of the non-compliance may include an additional dosage of the medication, intake of the medication prior to the threshold time associated with the dosage schedule of the medication, consumption of insufficient medication, and the like. The alerting engine 218 updates such non-compliance scenarios in the application 116 by providing the alert message.

Additionally, the alerting engine 218 may be configured to receive a plurality of alert customizable settings for customizing the alert message. The alert customizable settings may be provided by the user 102 based on providing user inputs in the application 116. The alert customizable settings include one of a type of the alert message, frequency of the alert message, and intensity of the alert message. The type of the alert message includes transmitting of the alert message in form of one of a visual signal or an audio signal to the users 102 and 106a-106c based on occurrence of the medication dispensing events as explained above. The visual signal corresponds to a UI displayed on the user devices 104, and 108a-108c for reminding the users 102 and 106. The intensity of the alert message corresponds to the intensity of the audio signal. The audio signal is played using speaker(s) or the microphone of the user devices 104, and 108a-108c based on occurrence of the medication dispensing events as explained above. Further, the frequency of the alert message corresponds to repetition of the alert message based on detection of the medication dispensing events. For instance, the frequency of the alert message may be set to 15 minutes by the user 102 or the users 106. In this scenario, the alerting engine 218 may transmit the alert message between the threshold time and the dosage schedule, or post the dosage schedule until expiry of the time interval for every 15 minutes, to remind the user 102 about the medication.

In one embodiment, the caregiver (e.g., the user 106b) and the health-care professional (e.g., the user 106c) may not use the application 116. In this scenario, the server system 200 may provide the alert message to the user devices 108b and 108c via the network 118 using messaging protocols (e.g., short message service (SMS)). It should be understood that the user 102 may provide mobile phone number of the users 106b and 106c.

In an embodiment, the server system 200 may transmit the alert message to the medication dispenser 114 based on occurrence of the medication dispensing events as explained above. The alert message enables a control unit of the medication dispenser 114 to illuminate light emitting diodes (LEDs) (see, 114b of FIG. 1) of the medication dispenser 114 to indicate the occurrence of each of the medication dispensing events. Further, in this scenario, the user 102 may provide the customizable alert settings related to color of illumination for each medication dispensing event.

The output computing engine 220 includes a suitable logic and/or interface for computing one or more parameters upon intake of the medication at each dosage schedule. More specifically, the control unit of the medication dispenser 114 is configured to transmit a quantifiable information, upon dispensing the medication at each dosage schedule. The quantifiable information may include, but not limited to, a time stamp of the medication intake, and a volume of the medication dispensed from the medication dispenser 114. The output computing engine 220 computes the parameters based on receipt of the quantifiable information. The parameters computed by the output computing engine 220 may include, but not limited to, a medication-instilling efficiency, a medication volume in the medication dispenser 114 after each dispensing, and number of days of remaining of the medication after each dispensing.

The medication dispenser 114 may be configured to dispense the medication in a controlled manner for a predefined time (e.g., 2 seconds) upon providing input on the button 114a. Thus, it should be understood that the medication-instilling efficiency is calculated based on the medication (e.g., ophthalmic liquid medication) dispensed on an eye (not shown in Figures) of the patient (such as the user 102) from the medication dispenser 114.

Further, the output computing engine 220 computes the medication volume and the number of days of medication remaining, upon each dispensing based at least on the initial volume of the medication present in the medication dispenser 114, the medication regimen and amount of medication dispensed from the medication dispenser 114 at the dosage schedule. Specifically, the number of days of medication remaining can be calculated as the ratio of the number of drops dispensed by the number of drops intended to be dispensed. The ratio is multiplied by the number of drops to be dispensed in the next day's regimen, yielding a realistic volume of medication required for the day. Further, the medication volume remaining for each subsequent day is summed until the sum of these days' volumes exceeds the calculated remaining volume. Furthermore, the output computing engine 220 calculates the remaining medication volume by subtracting the volume dispensed (number of drops actually dispensed multiplied by the drop volume) from the initial medication volume.

Upon computing the parameters, the server system 200 is configured to transmit the parameters to the user 102 and the authorized users 106a-106c to whom the user 102 authorized to share the information related to the medication usage. In other words, the server system 200 updates the parameters in the application 116 based on occurrence of each of the medication dispensing events. This allows the user 102 and the authorized users 106a-106c to monitor the medication usage of the user 102.

Additionally, the server system 200 may monitor a refill status of the medication present in the medication dispenser 114 based at least on the aforementioned parameters. Specifically, the server system 200 is configured to send a notification to the user 102 and the users 106a-106c (i.e. the caregiver(s) and the health-care professional(s)) based on determining the refill status of the medication. The server system 200 determines the refill status of the medication, based at least on the number of days remaining of the medication. Specifically, the server system 200 determines the refill status of the medication, if the number of days remaining of the medication is determined to be less than a predefined threshold value. Thereafter, the notification is transmitted to the users 102 and 106a-106c for allowing the user 102 to plan the medication refill/renewal accordingly. For instance, the number of days remaining of the medication may be 5 days and the predefined threshold value may be set as 6 days, in this scenario, the user 102 or the users 106a-106c are transmitted with the notification for allowing the user 102 to plan the medication refill accordingly. In an embodiment, the predefined threshold value may be set by the server system 200. In another embodiment, the predefined threshold value may be set by the user 106 or the users 106a-106c based by providing inputs in the application 116.

In one scenario, the user 102 may provide a refill request by providing inputs in the application 116 based on receipt of the notification. The refill request may include details related to the medication, prescription from the health-care professional (e.g., the user 106c), and the like. Upon receipt of the refill request, the application 116 may provide a UI depicting an online store associated with the application 116 managed by the server system 200 for placing the order for the medication. It should be understood that the user 102 is requested to select a payment option (e.g., cash on delivery (COD), payment card, online payment, unified payment interface (UPI), etc.) for placing the order for the medication. Further, the user 102 may be provided with delivery status of the medication upon placing the order. In one example scenario, the user 102 may initiate the refill, prior to receipt of the notification in the application 116.

In another scenario, the notification is sent directly to the health-care professional (e.g., the user 106c) of the user 102 for indicating the refill status of the medication as explained above. In this scenario, the user 106c who is the health-care professional of the user 102 is reminded with renewal of prescription associated with the medication of the user 102. In other words, the health-care professional (such as the user 106c) in charge of treating the user 102 (provided the user 102 has selected them as a health-care professional allowed to access their medication usage data) is notified that the prescription is due for renewal. In this case, the user 106c transmits a prescription renewal form to the server system 200, upon renewing the prescription of the medication by providing inputs in the application 116. The prescription renewal form may include details related to the medication (e.g., medication name, dosage, quantity etc.). This enables the server system 200 to place the order for the medication based on receipt of payment details from the user 102 as explained above.

In one example embodiment, the server system 200 is configured to receive a signal from the user device 104 of based at least on determining disconnection of the wireless communication between the user device 104 and the medication dispenser 114. In case of short-range communication, the disconnection of the wireless communication between the user device 104 and the medication dispenser 114 may be based on absence of a periodic input (e.g., for every 3 hours) from the control unit of the medication dispenser 114. Similarly, the server system 200 detects the disconnection of the wireless communication between the user device 104 and the medication dispenser 114 based on absence of the periodic input from the user device 104. The server system 200 transmits a warning message to the user device 104 to indicate the user 102 about the disconnection of the wireless communication between the user device 104 and the medication dispenser 114. This allows the user 102 to check an active status of the short range communication protocols in the user device 104 for reconnecting the user device 104 with the medication dispenser 114.

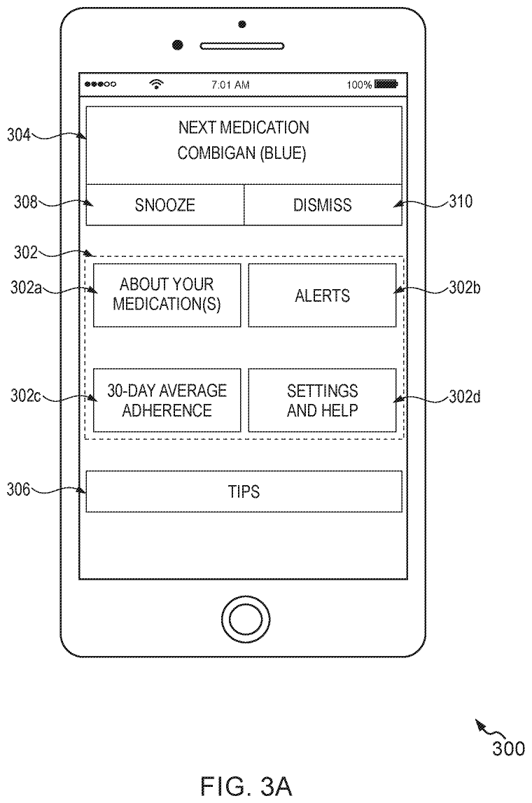

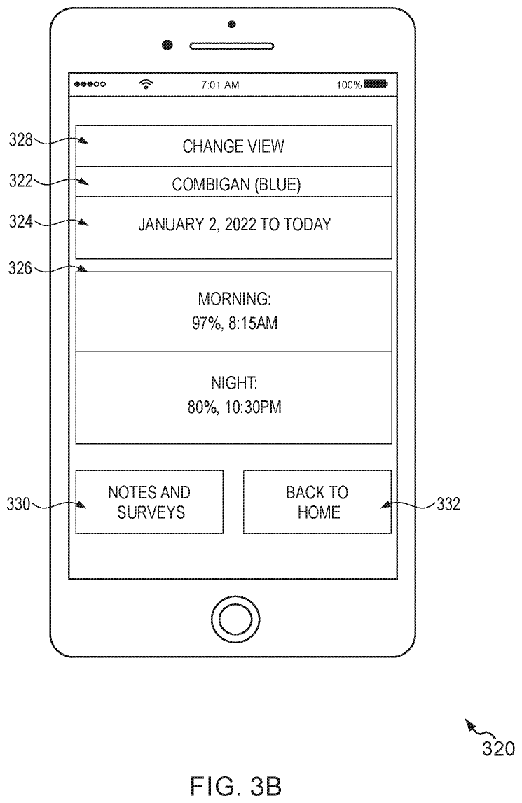

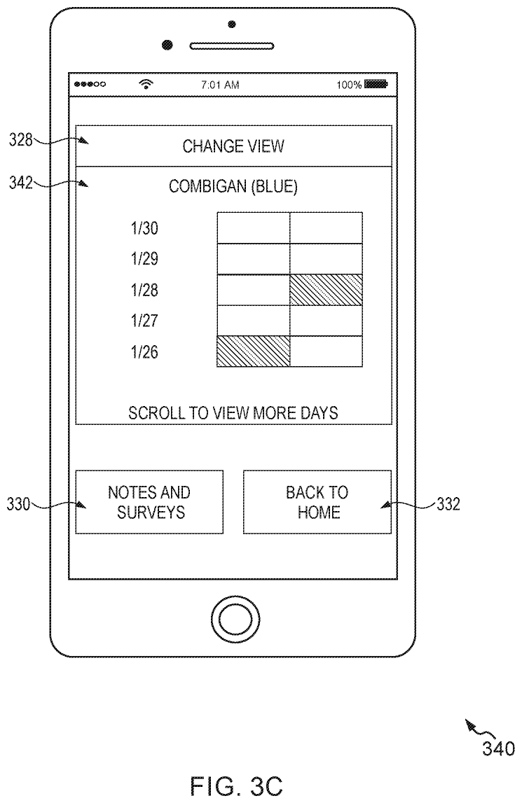

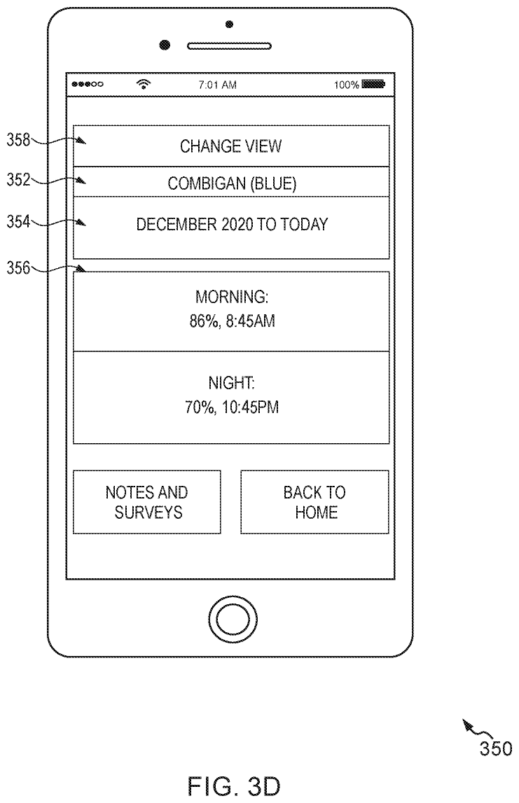

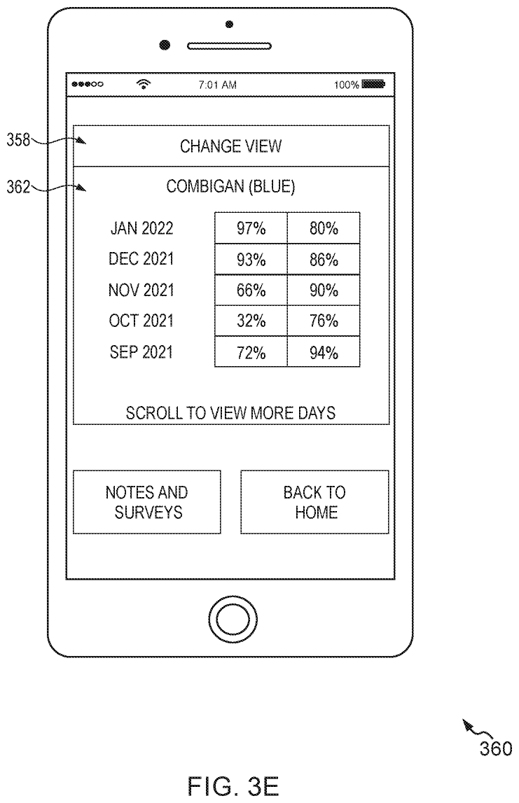

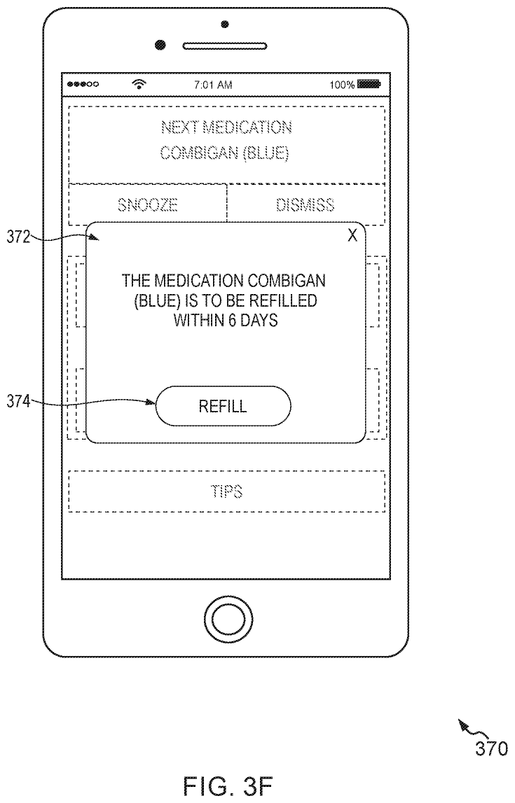

FIGS. 3A-3F collectively, represent example representation of user interfaces (UIs) displayed to the user 102 (i.e. the patient) for managing the medication usage, in accordance with an embodiment of the present disclosure.

Referring to FIG. 3A, illustrates an example representation of a UI 300 for monitoring the medication usage, in accordance with an example embodiment of the present disclosure. As shown, the UI 300 corresponds to a homepage of the application 116. The UI 300 is depicted to the user 102 based upon authentication of the login credentials associated with the user 102. The UI 300 is depicted to include a plurality of options 302, an alert message 304 and a tip option 306.

The plurality of options 302 includes an option 302a, 302b, 302c and 302d associated with the text “About your medication(s)”, “Alerts”, “30-Day average adherence”, “Settings and help”, respectively. The user 102 may provide input on the option 302a to monitor the medication usage of the user 102. Specifically, the user 102 may provide the user inputs in the application 116 such as the medication regimen, details of the medication, initial volume of the medication, the dosage schedules of the medication, and the like. Based on user selection of the option 302a, the user 102 is directed to a UI 320 for tracking the medication details of the user 102.

More specifically, the UI 320 is depicted to include a medication field 322, a medication regimen field 324 and a dosage schedule 326. The medication field 322 is depicted to include details related to the medication (exemplary depicted to be “Combigan (Blue)”) that is to be taken by the user 102. Further, the medication regimen field 324 depicts details related to a course period associated with the medication for treating the infection of the eye. It is evident that the course period is depicted to be on a daily basis (exemplarily depicted to be “Jan. 2, 2022 to today” in FIG. 3B). Further, the dosage schedules 326 associated with the medication are exemplarily depicted to be “Morning: 97%, 8:15 am” and “Night: 80%, 10:30 pm”. Further, the exemplary values such as “97%” and “80%” depicted in the dosage schedules 326 represent the adherence to each dosage. It should be understood by a person skilled in the art that the user 102 is required to take the medication two times (i.e. morning and evening) daily until completion of the course period. The user 102 may view the consumption details of the medication in a tabular column based on user input on a button 328 associated with the text “Change view”. Based on user input on the button 328, the user 102 is directed to a UI 340 depicted to include a tabular column 342 (as shown in FIG. 3C). Each row of the tabular column 342 is associated with a particular day of the course period (as shown in FIG. 3C). As such, based on consumption and missed dosages of the medication as per the dosage schedule 326, the tabular column 342 is updated. For illustrative purpose, the missed dosage schedules are depicted using hatching in the tabular column 342.

Further, the user 102 may record notes and/or take surveys in the application 116 based on user input on a button 330 (as shown in FIG. 3B). For instance, the user 102 may note symptoms, side effects experienced by the user 102 based on taking the medication. The user 102 may be redirected to the UI 300 based on providing user input on a button 332 (as shown in FIG. 3B).

In an embodiment, the course period associated with the medication may be depicted as a monthly basis (see, UI 350 of FIG. 3D). It should be understood that the user selection related to the depiction of the course period in the application 116 may be provided by the user 102 based on user input on the option 302d. The user 102 may be provided with a UI (not shown in Figures) for providing inputs related to the course period. As shown, the UI 350 is depicted to include a medication field 352 depicted to include details related to the medication (exemplarily depicted to be “Combigan (Blue)”). Further, the UI 350 includes a medication regimen field 354 depicting a course period on the monthly basis (exemplarily depicted to be “December 2020 to today”). Furthermore, the UI 350 depicts a dosage schedule 356 associated with the medication (exemplarily depicted to be “Morning: 86%, 8:45 am” and “Night: 70%, 10:45 pm”. The user 102 may view the consumption details of the medication in a tabular column based on user input on a button 358 associated with the text “Change view”. Based on user input on the button 358, the user 102 is directed to a UI 360 depicted to include a tabular column 362 (as shown in FIG. 3E). Each row of the tabular column 362 is associated with a particular month of the course period (as shown in FIG. 3E). As such, based on consumption and missed dosages of the medication as per the dosage schedules 326, each month in the tabular column 362 is updated. Specifically, the server system 200 may compute an average through the month based at least on the intake of the medication as per the dosage schedule 356 and update the tabular column 362.

Further, the user 102 receives the alert message 304 based on occurrence of the medication dispensing events as explained above. In one example scenario, the user 102 may receive the alert message 304 at the threshold time, prior to the dosage schedule associated with the medication, for reminding the user 102 to take the medication (as shown in FIG. 3A). It is evident that the alert message 304 is provided as the visual signal in the application 116. Additionally, the alert message 304 may be played using the speaker of the user device 104 as explained with reference to FIG. 2. The user 102 may either snooze or dismiss the alert message 304 based on providing input on a button 308 or a button 310, respectively. Additionally, the user 102 may track the alert messages received based on occurrence of the various medication dispensing events by providing the user input on the option 302b. Based on providing the user input on the option 302b, the user 102 may be directed to a UI (not shown in Figures) which lists out the alert message received at each dispensing event.

Further, the user 102 may track details associated with a medication regimen adherence based on providing input on the option 302c. For instance, the user 102 may track 30-day average adherence of the medication. The user 102 may receive regular feedbacks from the health-care professional(s) (e.g., the user 106c) of the user 102 when they adhere to their regimen. The user 102 may access or view the feedbacks by providing input on the tip option 306. As explained above, the user 102 may share the medication usage data to the authorized users 106a-106c of the application 116 and customize the alert messages, and the like as explained with reference to FIG. 2. As such, the user 102 may provide details of the users 106 and the alert customizable settings by selecting the option 302d.

As explained above, the server system 200 transmits the notification to the user device 104 based on tracking the refill status of the medication (see, 370 of FIG. 3F. As shown, the UI 370 is depicted to include a notification 372 exemplarily depicted to be “The medication Combigan (Blue) is to be refilled within 6 days”. The user 102 may provide input on a button 374 associated with the text “REFILL” for ordering the medication from the online store of the application 116. Based on the user input on the button 374, the user 102 may be provided with a UI depicting a refill request for receiving inputs related to the medication, prescription, and the like which are already explained with reference to FIG. 2, and therefore it is not reiterated.

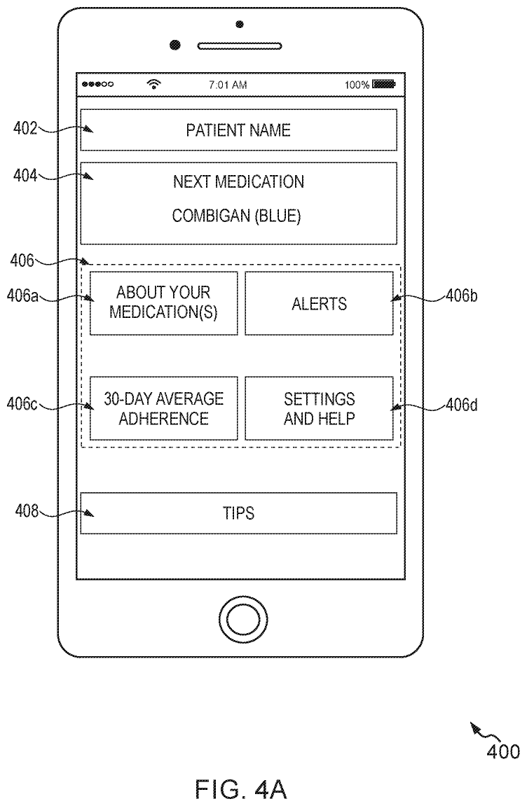

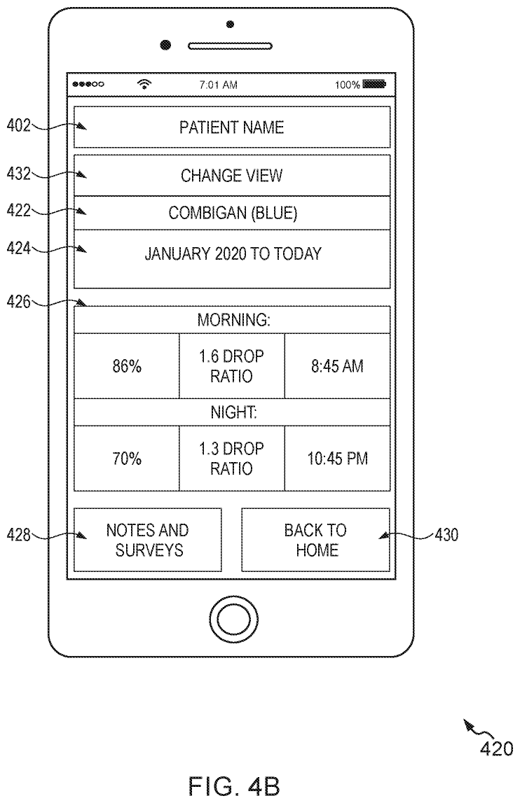

FIGS. 4A-4B collectively, represent example representation of user interfaces (UIs) displayed to the caregiver (e.g., the user 106a or the user 106b) of the user 102 for managing the medication usage of the user 102, in accordance with an embodiment of the present disclosure.

Referring to FIG. 4A, it illustrates an example representation of a UI 400, in accordance with an example embodiment of the present disclosure. As shown, the UI 400 is depicted to include a homepage of the application 116. The UI 400 is provided for the caregiver for accessing the medication usage of the user 102. As explained above, the medication usage of the user 102 is transmitted to the caregiver (e.g., the user 106a) based on the user 102 authorization to share the medication usage as explained with reference to FIG. 2.

The UI 400 is depicted to include a patient name 402, an alert message 404, a plurality of options 406 and a tip option 408. As explained in FIG. 3A, the alert message 404 is similar to the alert message 304 which is exemplarily depicted to include the alert related to next medication of the user 102. Further, the user 106a accesses the data related to medication(s) of the user 102, alerts received based on occurrence of the medication dispensing events and a 30-day average adherence data associated with the medication consumption by the user 102 for 30 days based on providing input on options 406a, 406b, and 406c, respectively. Based on providing input on the option 406a, the user 106a is directed to a UI 420 (as shown in FIG. 4B) for accessing the medication data of the user 102. The UI 420 is depicted to include the patient name 402, a medication field 422, a medication regimen field 424 and a dosage schedule 426.

The medication field 422 is depicted to include the details of the medication, the medication regimen field 424 depicts the course period and the dosage schedule 426. The dosage schedule 426 depicts an average time for each dose in a day, drop efficiency, and the adherence (as shown in FIG. 4B). The user 106a may access the notes and surveys of the user 102 by providing input on a button 428. The user 106a may be redirected to the home page (i.e. the UI 400) based on user input on a button 430. Further, the user 106a may view the medication usage data in a tabular column (similar to the tabular columns 342 and 362 shown in the UI 340 and the UI 360, respectively) by providing input on a button 432 associated with the text “Change view”. It should be noted that the UI 420 is similar to the UI 320, therefore, the UI 420 is herein not explained in greater detail. The user 106a may be provided tips, educational content about the medication and access notes and surveys of the user 102 by providing input on the tip option 408. Moreover, the user 106a may provide general settings such as inputs related to minimum period of dosage times to the user 102 through the application 116 by providing input on an option 406d.

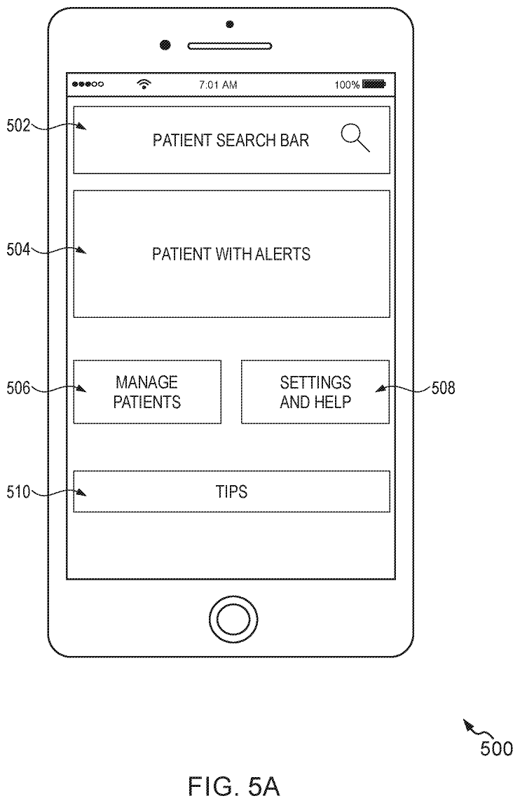

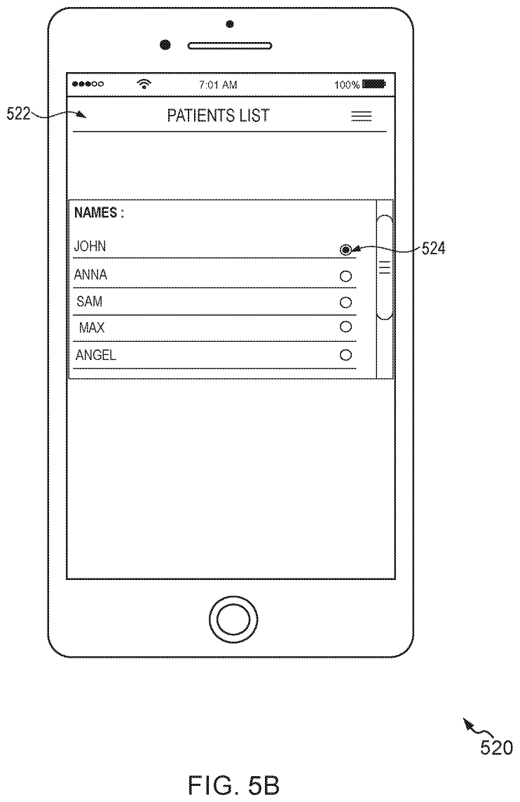

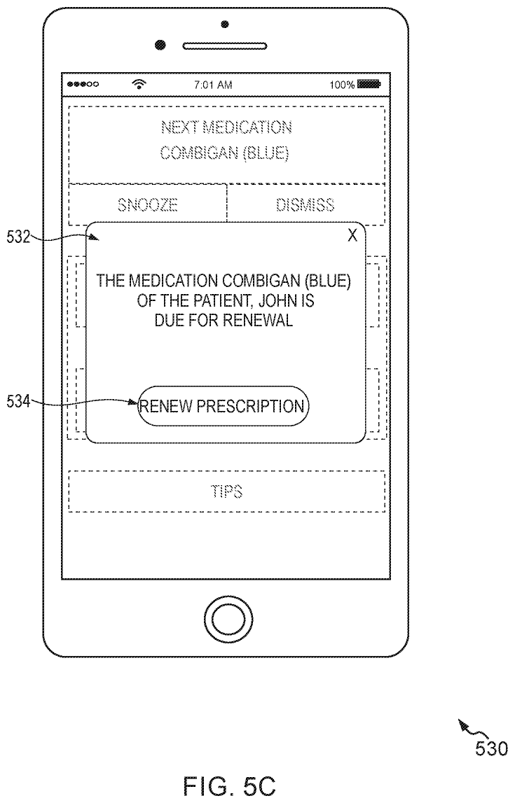

FIGS. 5A-5C collectively, represent example representations of user interfaces (UIs) displayed to the health-care professional (e.g., the user 106c) of the user 102 for managing the medication usage of the user 102, in accordance with an embodiment of the present disclosure.

Referring to FIG. 5A, it illustrates an example representation of a UI 500, in accordance with an example embodiment of the present disclosure. As shown, the UI 500 is depicted to include a homepage of the application 116. The UI 500 is provided for the health-care professional for accessing the medication usage of the patients (e.g., the user 102). As explained above, the medication usage of the user 102 is transmitted to the health-care professional (e.g., the user 106c) based on the user 102 authorization to share the medication usage as explained with reference to FIG. 2.

The UI 500 is depicted to include an option 506 associated with the text “Manage patients”. The user 106c is provided with a UI 520 that includes a list of patients 522 who are treated by the user 106c (as shown in FIG. 5B) based on providing input on the option 506. The user 106c may select a patient from the list of patients to view the medication usage. For instance, the user 106c may select a patient (exemplarily depicted to be “JOHN”) from the list of patients 522 for monitoring the medication usage. It should be noted that the user 106c may select the patient by providing input on a radio button (see, 524 of FIG. 5B). The patient exemplarily selected to be “JOHN” may be the user 102. Based on user selection, the user 106c is rendered with a UI similar to the UI 420 in the application 116 for accessing the medication usage of the user 102. As explained above, the user 106c may view the medication details, the course period and the dosage schedule (e.g., the medication time, volume of medication). Also, the user 106c may view the medication usage data in a tabular column (similar to the tabular columns 342 and 362) as explained above. In one scenario, the user 106c may provide inputs such as a name of the patient (e.g., the user 102) in a search field 502 depicted in the UI 500 for searching the patient to access the medication usage of the patient.

Further, the user 106c manages alerts and notifications associated with each patient of the user 106c by providing input on an option 504 depicted in the UI 500. In an example scenario, the user 106c may receive a notification related to refilling of the medication associated with the user 102 (as shown in FIG. 5C). In other words, the user 106c is notified that the prescription for the medication associated with the user 102 is due for renewal. As shown in FIG. 5C, a UI 530 is depicted to include a notification 532 exemplarily depicted to be “The medication Combigan (Blue) of the patient, JOHN is due for renewal”. The user 106c renews the prescription by providing input on a button 534 associated with the text “RENEW PRESCRIPTION”. Upon providing the input on the button 534, the prescription renewal form will be transmitted to the server system 200 as explained with reference to FIG. 2. Additionally, the user 106c may be provided tips, educational content about the medication and access notes and surveys of the user 102 by providing input on an option 510. The user 106c may provide general settings such as inputs related to minimum period of dosage times to the user 102 through the application 116 by providing input on an option 508.

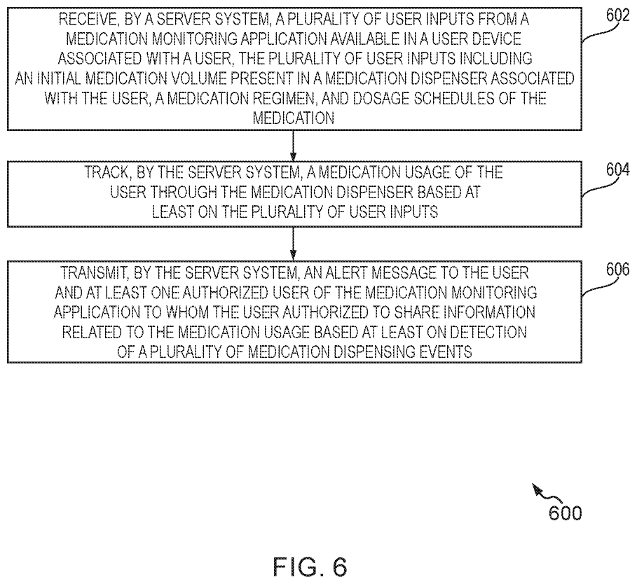

FIG. 6 illustrates a flow diagram of a computer-implemented method 600 for managing the medication usage of the user 102 and providing alerts to at least the user 102 and the users 106a-106c based upon occurrence of the medication dispensing events, in accordance with an embodiment of the present disclosure. The method 600 depicted in the flow diagram may be executed by, for example, the server system 110 or the server system 200. Operations of the flow diagram of method 600, and combinations of operation in the flow diagram of method 600, may be implemented by, for example, hardware, firmware, a processor, circuitry, and/or a different device associated with the execution of software that includes one or more computer program instructions. It is noted that the operations of the method 600 can be described and/or practiced by using a system other than these server systems. The method 600 starts at operation 602.

At operation 602, the method 600 includes receiving, by a server system, a plurality of user inputs from a medication monitoring application available in a user device associated with a user. The plurality of user inputs includes an initial medication volume present in a medication dispenser associated with the user, a medication regimen, and dosage schedules of the medication. The medication dispenser 114 is communicably coupled with the user device 104 or with the server system 200 as explained above.

At operation 604, the method 600 includes tracking, by the server system, a medication usage of the user through the medication dispenser based at least on the plurality of user inputs.

At operation 606, the method 600 includes transmitting, by the server system, an alert message based on detection of a plurality of medication dispensing events to the user and at least one authorized user of the medication monitoring application to whom the user authorized to share information related to the medication usage. The plurality of medication dispensing events is detected based at least on the plurality of user inputs.

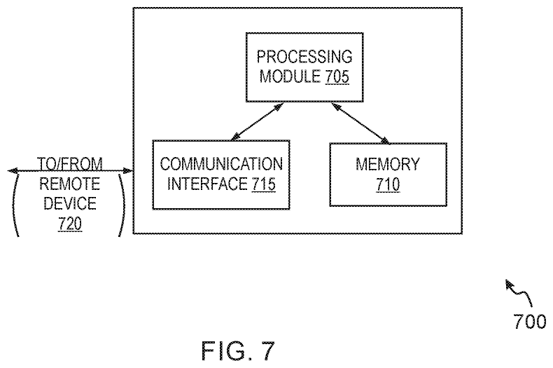

FIG. 7 illustrates a simplified block diagram representation of a control unit 700 of the medication dispenser 114, in accordance with an example embodiment of the present disclosure. The control unit 700 includes at least one processing module 705, a memory 710, and a communication interface 715. The one or more components of the control unit 700 communicate with each other.

It is noted that although the control unit 700 is depicted to include only one processing module, the control unit 700 may include more number of processors therein. In an embodiment, the memory 710 is capable of storing executable instructions. Further, the processing module 705 is capable of executing the platform instructions to perform the operations described herein. In an embodiment, the processing module 705 may be embodied as a multi-core processor, a single core processor, or a combination of one or more multi-core processors and one or more single core processors.

The memory 710 may be embodied as one or more volatile memory devices, one or more non-volatile memory devices, and/or a combination of one or more volatile memory devices and non-volatile memory devices. Examples of the memory 710 include a random-access memory (RAM), a read-only memory (ROM), a removable storage drive, and the like.

The processing module 705 is configured to communicate with a remote device 720 via the communication interface 715. Examples of the remote device 720 include the server system 200 or the server system 110, the user device 104, a cloud computing system (not shown in Figures) and the like. The processing module 705 of the control unit 700 is configured to transmit the quantifiable information (such as the time stamp of the medication intake, the volume of the medication dispensed from the medication dispenser 114) to the server system 200 after each dispensing. Further, the processing module 705 is configured to communicate with the application 116 on a periodic basis via the communication interface 715. Furthermore, the processing module 705 illuminates the LED (see, 114b of FIG. 1) of the medication dispenser 114 based on receipt of the alert message upon occurrence of the medication dispensing events. The processing module 705 is configured to operate the medication dispenser 114 to dispense the medication in a controlled manner. Further, the one or more steps performed by the control unit 700 are already explained above, and therefore they are not reiterated herein, for the sake of brevity.

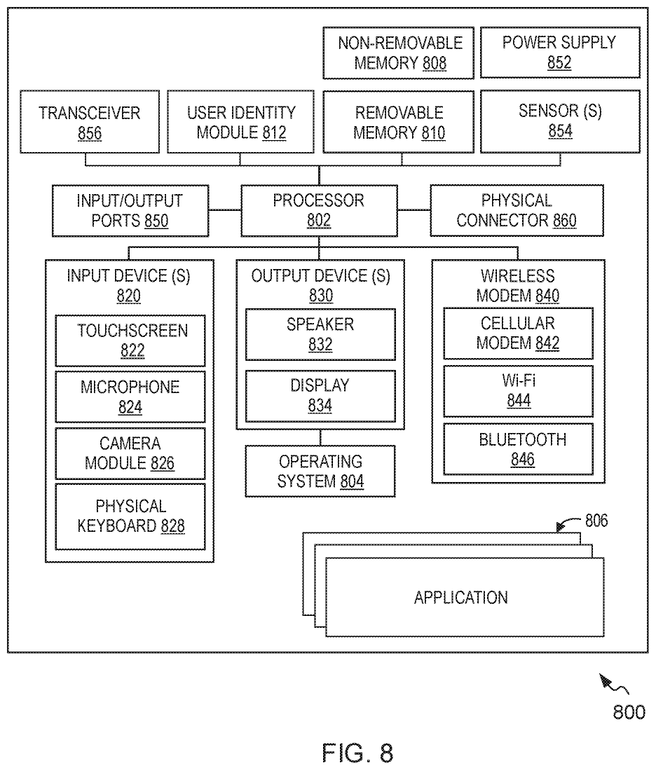

FIG. 8 is a simplified block diagram of an electronic device 800 capable of implementing various embodiments of the present disclosure. For example, the electronic device 800 may correspond to the user devices 104 and 108a-108c of FIG. 1. The electronic device 800 is depicted to include one or more applications 806. For example, the one or more applications 806 may include the application 116 of FIG. 1. The application 116 can be an instance of a medication monitoring application downloaded from the server system 110 or the server system 200. One of the one or more applications 806 installed on the electronic device 800 are capable of communicating with a server system for monitoring the medication usage of patient and providing alerts to the patient, and caregiver(s) and health-care professional(s) of the patient.

It should be understood that the electronic device 800 as illustrated and hereinafter described is merely illustrative of one type of device and should not be taken to limit the scope of the embodiments. As such, it should be appreciated that at least some of the components described below in connection with the electronic device 800 may be optional and thus in an embodiment may include more, less or different components than those described in connection with the embodiment of the FIG. 8. As such, among other examples, the electronic device 800 could be any of a mobile electronic device, for example, cellular phones, tablet computers, laptops, mobile computers, personal digital assistants (PDAs), mobile televisions, mobile digital assistants, or any combination of the aforementioned, and other types of communication or multimedia devices.

The illustrated electronic device 800 includes a controller or a processor 802 (e.g., a signal processor, microprocessor, ASIC, or other control and processing logic circuitry) for performing such tasks as signal coding, data processing, image processing, input/output processing, power control, and/or other functions. An operating system 804 controls the allocation and usage of the components of the electronic device 800 and supports for one or more operations of the application (see, the applications 806), such as the application 116 that implements one or more of the innovative features described herein. In addition, the applications 806 may include common mobile computing applications (e.g., telephony applications, email applications, calendars, contact managers, web browsers, messaging applications) or any other computing application.

The illustrated electronic device 800 includes one or more memory components, for example, a non-removable memory 808 and/or removable memory 810. The non-removable memory 808 and/or the removable memory 810 may be collectively known as a database in an embodiment. The non-removable memory 808 can include RAM, ROM, flash memory, a hard disk, or other well-known memory storage technologies. The removable memory 810 can include flash memory, smart cards, or a Subscriber Identity Module (SIM). The one or more memory components can be used for storing data and/or code for running the operating system 804 and the applications 806. The electronic device 800 may further include a user identity module (UIM) 812. The UIM 812 may be a memory device having a processor built in. The UIM 812 may include, for example, a subscriber identity module (SIM), a universal integrated circuit card (UICC), a universal subscriber identity module (USIM), a removable user identity module (R-UIM), or any other smart card. The UIM 812 typically stores information elements related to a mobile subscriber. The UIM 812 in form of the SIM card is well known in Global System for Mobile (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The electronic device 800 can support one or more input devices 820 and one or more output devices 830. Examples of the input devices 820 may include, but are not limited to, a touch screen/a display screen 822 (e.g., capable of capturing finger tap inputs, finger gesture inputs, multi-finger tap inputs, multi-finger gesture inputs, or keystroke inputs from a virtual keyboard or keypad), a microphone 824 (e.g., capable of capturing voice input), a camera module 826 (e.g., capable of capturing still picture images and/or video images) and a physical keyboard 828. Examples of the output devices 830 may include, but are not limited to, a speaker 832 and a display 834. Other possible output devices can include piezoelectric or other haptic output devices. Some devices can serve more than one input/output function. For example, the touch screen 822 and the display 834 can be combined into a single input/output device.

A wireless modem 840 can be coupled to one or more antennas (not shown in FIG. 8) and can support two-way communications between the processor 802 and external devices, as is well understood in the art. The wireless modem 840 is shown generically and can include, for example, a cellular modem 842 for communicating at long range with the mobile communication network, a Wi-Fi compatible modem 844 for communicating at short range with an external Bluetooth-equipped device or a local wireless data network or router, and/or a Bluetooth-compatible modem 846. The wireless modem 840 is typically configured for communication with one or more cellular networks, such as a GSM network for data and voice communications within a single cellular network, between cellular networks, or between the electronic device 800 and a public switched telephone network (PSTN).

The electronic device 800 can further include one or more input/output ports 850, a power supply 852, one or more sensors 854 for example, an accelerometer, a gyroscope, a compass, or an infrared proximity sensor for detecting the orientation or motion of the electronic device 800 and biometric sensors for scanning biometric identity of an authorized user, a transceiver 856 (for wirelessly transmitting analog or digital signals) and/or a physical connector 860, which can be a USB port, IEEE 1294 (FireWire) port, and/or RS-232 port. The illustrated components are not required or all-inclusive, as any of the components shown can be deleted and other components can be added.

The disclosed method with reference to FIG. 7, or one or more operations of the server system 200 may be implemented using software including computer-executable instructions stored on one or more computer-readable media (e.g., non-transitory computer-readable media, such as one or more optical media discs, volatile memory components (e.g., DRAM or SRAM), or non-volatile memory or storage components (e.g., hard drives or solid-state non-volatile memory components, such as Flash memory components)) and executed on a computer (e.g., any suitable computer, such as a laptop computer, net book, Web book, tablet computing device, smart phone, or other mobile computing device). Such software may be executed, for example, on a single local computer or in a network environment (e.g., via the Internet, a wide-area network, a local-area network, a remote web-based server, a client-server network (such as a cloud computing network), or other such network) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such a suitable communication means includes, for example, the Internet, the World Wide Web, an intranet, software applications, cable (including fiber optic cable), magnetic communications, electromagnetic communications (including RF, microwave, and infrared communications), electronic communications, or other such communication means.

Although the invention has been described with reference to specific exemplary embodiments, it is noted that various modifications and changes may be made to these embodiments without departing from the broad spirit and scope of the invention. For example, the various operations, blocks, etc., described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application specific integrated circuit (ASIC) circuitry and/or in Digital Signal Processor (DSP) circuitry).

Particularly, the server system 200 and its various components may be enabled using software and/or using transistors, logic gates, and electrical circuits (for example, integrated circuit circuitry such as ASIC circuitry). Various embodiments of the invention may include one or more computer programs stored or otherwise embodied on a computer-readable medium, wherein the computer programs are configured to cause a processor or computer to perform one or more operations. A computer-readable medium storing, embodying, or encoded with a computer program, or similar language, may be embodied as a tangible data storage device storing one or more software programs that are configured to cause a processor or computer to perform one or more operations. Such operations may be, for example, any of the steps or operations described herein. In some embodiments, the computer programs may be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), DVD (Digital Versatile Disc), BD (BLU-RAY® Disc), and semiconductor memories (such as mask ROM, PROM (programmable ROM), EPROM (erasable PROM), flash memory, RAM (random access memory), etc.). Additionally, a tangible data storage device may be embodied as one or more volatile memory devices, one or more non-volatile memory devices, and/or a combination of one or more volatile memory devices and non-volatile memory devices. In some embodiments, the computer programs may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

Various embodiments of the disclosure, as discussed above, may be practiced with steps and/or operations in a different order, and/or with hardware elements in configurations, which are different than those which are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A computer-implemented method, comprising:
receiving, by a server system, a plurality of user inputs from a medication monitoring application available in a user device associated with a user, the plurality of user inputs comprising an initial medication volume present in a medication dispenser associated with the user, a medication regimen, and dosage schedules of a medication;
tracking, by the server system, a medication usage of the user through the medication dispenser based at least on the plurality of user inputs; and
transmitting, by the server system, an alert message to the user and at least one authorized user of the medication monitoring application to whom the user authorized to share information related to the medication usage based at least on detection of a plurality of medication dispensing events.

2. The computer-implemented method as claimed in claim 1, wherein transmitting the alert message upon occurrence of the plurality of medication dispensing events comprises:
transmitting the alert message to the user at a threshold time, prior to a dosage schedule of the medication for reminding the user to take the medication; and
transmitting the alert message to at least the user and the at least one authorized user post the dosage schedule of the medication, if the dosage schedule has passed and upon detection of non-compliance with the dosage schedules of the medication associated with the user.

3. The computer-implemented method as claimed in claim 2, further comprising:
receiving, by the server system, a plurality of alert customizable settings for customizing the alert message based on user inputs in the medication monitoring application, wherein the plurality of alert customizable settings comprises one of a type of the alert message, frequency of the alert message, and intensity of the alert message.

4. The computer-implemented method as claimed in claim 3, wherein the type of the alert message being transmitted to at least the user and the at least one authorized user of the medication monitoring application is in form of one of a visual signal and an audio signal.

5. The computer-implemented method as claimed in claim 1, wherein the medication dispensed from the medication dispenser is an ophthalmic liquid medication.

6. The computer-implemented method as claimed in claim 1, wherein the at least one authorized user of the medication monitoring application to whom the user authorized to share the information related to the medication usage is one of a health care professional and caregivers of the user.

7. The computer-implemented method as claimed in claim 1, further comprising:
receiving, by the server system, a quantifiable information related to a time stamp of the medication intake, and a volume of the medication dispensed from the medication dispenser, upon intake of the medication at each dosage schedule;
computing, by the server system, one or more parameters related to a medication-instilling efficiency, a medication volume in the medication dispenser after each dispensing, and number of days remaining of the medication; and
transmitting, by the server system, the one or more parameters to the medication monitoring application through the medication dispenser, for allowing the user and the at least one authorized user to monitor the medication usage of the user.

8. The computer-implemented method as claimed in claim 7, further comprising:
sending, by the server system, a notification to the user and the at least one authorized user of the medication monitoring application, if the number of days remaining of the medication is determined to be less than a predefined threshold value; and
receiving, by the server system, one of a refill request from the user and a prescription renewal form from the at least one authorized user for refilling the medication associated with the user.

9. The computer-implemented method as claimed in claim 1, further comprising:
receiving, by the server system, login credentials associated with the user for authenticating the user to access the medication monitoring application to monitor the medication usage; and
facilitating, by the server system, a wireless communication between the medication monitoring application and a control unit of the medication dispenser using short range communication protocols, based upon entering a unique code associated with the medication dispenser in the medication monitoring application.

10. The computer-implemented method as claimed in claim 9, further comprising:
receiving, by the server system, a signal from the user device based at least on determining disconnection of the wireless communication between the user device and the medication dispenser, wherein the signal is transmitted by the user device due to absence of a periodic input from the control unit of the medication dispenser; and
transmitting, by the server system, a warning message to the user device to indicate the disconnection of the wireless communication between the user device and the medication dispenser, thereby allowing the user to check an active status of the short range communication protocols in the user device for reconnecting the user device with the medication dispenser.

11. A server system, comprising:
a communication interface;
a memory storing executable instructions; and
a processor operatively coupled with the communication interface and the memory, the processor configured to execute the executable instructions to cause the server system to at least:
receive a plurality of user inputs from a medication monitoring application available in a user device associated with a user, the plurality of user inputs comprising an initial medication volume present in a medication dispenser associated with the user, a medication regimen, and dosage schedules of a medication,
track a medication usage of the user through the medication dispenser based at least on the plurality of user inputs, and
transmit an alert message to the user and at least one authorized user of the medication monitoring application to whom the user authorized to share information related to the medication usage based at least on detection of a plurality of medication dispensing events.

12. The server system as claimed in claim 11, wherein the server system is further caused to:
transmit the alert message to the user at a threshold time, prior to a dosage schedule of the medication for reminding the user to take the medication; and
transmit the alert message to at least the user and the at least one authorized user post the dosage schedule of the medication, if the dosage schedule has passed and upon detection of non-compliance with the dosage schedules of the medication associated with the user.

13. The server system as claimed in claim 12, wherein the server system is further caused to:
receive a plurality of alert customizable settings for customizing the alert message based on user inputs in the medication monitoring application, wherein the plurality of alert customizable settings comprises one of a type of the alert message, frequency of the alert message, and intensity of the alert message.

14. The server system as claimed in claim 13, wherein the type of the alert message being transmitted to at least the user and the at least one authorized user of the medication monitoring application is in form of one of a visual signal and an audio signal.

15. The server system as claimed in claim 11, wherein the medication dispensed from the medication dispenser is an ophthalmic liquid medication.

16. The server system as claimed in claim 11, wherein the at least one authorized user of the medication monitoring application to whom the user authorized to share the information related to the medication usage is one of a health care professional and caregivers of the user.

17. The server system as claimed in claim 11, wherein the server system is further caused to:
receive a quantifiable information related to a time stamp of the medication intake, and a volume of the medication dispensed from the medication dispenser, upon intake of the medication at each dosage schedule;
compute one or more parameters related to a medication-instilling efficiency, a medication volume in the medication dispenser after each dispensing, and number of days remaining of the medication; and
transmit the one or more parameters to the medication monitoring application through the medication dispenser, for allowing the user and the at least one authorized user to monitor the medication usage of the user.

18. The server system as claimed in claim 17, wherein the server system is further caused to:
send a notification to the user and the at least one authorized user of the medication monitoring application, if the number of days remaining of the medication is determined to be less than a predefined threshold value; and
receive one of a refill request from the user and a prescription renewal form from the at least one authorized user for refilling the medication associated with the user.

19. The server system as claimed in claim 11, wherein the server system is further caused to:
receive login credentials associated with the user for authenticating the user to access the medication monitoring application to monitor the medication usage; and
facilitate a wireless communication between the medication monitoring application and a control unit of the medication dispenser using short range communication protocols, based upon entering a unique code associated with the medication dispenser in the medication monitoring application.

20. The server system as claimed in claim 19, wherein the server system is further caused to:
receive a signal from the user device based at least on determining disconnection of the wireless communication between the user device and the medication dispenser, wherein the signal is transmitted by the user device due to absence of a periodic input from the control unit of the medication dispenser; and
transmit a warning message to the user device to indicate the disconnection of the wireless communication between the user device and the medication dispenser, thereby allowing the user to check an active status of the short range communication protocols in the user device for reconnecting the user device with the medication dispenser.
